(19) United States
(12) Patent Application Publication
(10) Pub. No.: US 2002/0034760 A1
(43) Pub. Date: Mar. 21, 2002

(54) Combination and method presenting and utilizing DNA analysis and for diagnosis and treatment

(76) Inventors: George Kindness, Middletown, OH (US); Brooke Schumm III, Ellicott City, MD (US); Andrew J. Walter, Glyndon, MD (US)

Correspondence Address: BROOKE SCHUMM, III, DANEKER, MCINTIRE, SCHUMM, PRINCE, GOLDSTEIN, ET A, 210 N CHARLES ST, SUITE 800, BALTIMORE, MD, 21201, US

(21) Appl. No.: 09/934,995

(22) Filed: Aug. 21, 2001

Related U.S. Application Data
(60) Provisional application No. 60/227,151, filed on Aug. 22, 2000.

(51) Int. Cl. C12Q 1/68; G06F 19/00; G01N 33/48; G01N 33/50

(52) U.S. Cl. 435/6; 702/20

(57) ABSTRACT

The invention proposes to examine DNA sequences, particularly glutathione-S-transferase (GST) sequences and cytochrome P450 (CYP450) sequences, and determine a patient's propensity to redox imbalance. Prophylactic enhancement of the glutathione pathway is proposed with or without additional tests. Additionally, storage of DNA sequences on electronic media for analysis of propensity to disease and comparison with non-defective sequences, and appropriate treatment is set out. An artificial intelligence algorithm to enable integration and development of one or more databases to enable diagnosis and treatment is part of the invention. References based on DNA sequences, particularly defective sequences in an electronic media database with hyperlinks to information on such defects is referenced.

This invention is a continuation-in-part of Provisional Application 60/227,151 filed Aug. 22, 2000, Provisional Application 60/297,816 filed Jun. 13, 2001, and Provisional Application 60/263,486 filed Jan. 23, 2001, and a provisional application of this name filed on the same day as this application, which provisional applications are incorporated by reference.

### SUMMARY OF INVENTION

After a patient or client has had all or critical DNA sequences determined, the inventors propose a system of proposed treatment The invention proposed to accomplish a determination by a combination of machines and instruments. The invention proposes to compare a client's or patient's genotypic expression reflected in the DNA sequences with databases of genotypic expression associated with various diseases and afflictions. After such comparison, if or when propensities to diseases or afflictions are determined, the invention proposes to investigate the glutathione pathway functionality and redox imbalance to determine further treatment, and then to administer treatment, particularly treatment by cystine, to restore proper glutathione levels. Other treatments for other chemical cycle imbalance may be indicated as well.

For further clarification of a propensity based on a genotypic expression, DNA sequence determination among relatives can be helpful. Samples could be taken immediately after death in conjunction with funeral arrangements for the purposes of determining a propensity to disease and risk factors of disease based on detected sequences synonymous or similar to known sequences or for propensities and risk factors statistically apparent based on manifested symptoms in such relatives. By taking a sample of a baby at birth, and taking a sample later in life, and comparing those samples to parent DNA, a determination can be made if a critical DNA change occurred during a person's life time or was inherited. Determination can be made of whether an environmental factor may have or may be causing or aggravating genotypic potential.

The first step is to have DNA sequence analysis performed of DNA samples.

### Description and Presentation of Data

Upon completion of DNA sequencing, the inventors propose to compile the information into electronically readable profiles stored in a form accessible by a general purpose computer. Those profiles would be electronically scanned for sequences of specific alleles. Those specific sequences from the selected profile would be compared to data bases with known sequences of alleles reflecting potential risks or actual alleles found with respect to specific diseases or afflictions. The analysis would be delivered to the client electronically or in other electronic media form such as CD-ROM or disk. A hard copy of key data would be prepared. The invention includes placing data on a CD-ROM in a form compatible for interface with a variety of software. Translation software to make the data compatible with outside programs would be included if need on the electronic media delivered, or a hyperlink to where a patient, client or health care provider could purchase such software would be included. A patient, on a personal computer, could view key or selected data and print a hard copy of key DNA profiles.

From the analysis, where there were matching sequences to known sequences indicating a propensity(ies) of disease or affliction, hyperlinks would be generated from a second data base corresponding to the DNA genetic markers to enable a client to obtain further information. The database can on a compact disk (CD), or alternatively, accessible by electronic means, usually through the Internet. These hyperlinks could be generated for two purposes. The first is to enable access to general information understandable by a layperson. The second is to enable access by a more sophisticated client or his health care provider to either other data bases or detailed literature.

Another feature of the electronic delivery system would be to have a security system so only the client and the client's selected personal choices could access the sequence information delivered. The proposed electronic delivery system would have a password system and software either programmed on electronic media or separately communicated to a client so that access to electronic media would only be by password. The method would alternatively contemplate transmission of the password separately from such electronic media by a postcard or independent secure electronic communication. A client could also direct transmission of data to his health care provider, and a password could be provided to a health care provider to enable that person to have unique access.

### APPLICATION

While reference is directly made in this disclosure to use in humans and persons, wherever such expression is used, it shall mean application to animals and in particular veterinary science. The claims are not limited to human analysis or therapy.

A positive application of the power of analysis of such DNA sequences is a method of analysis and consequent administration of appropriate drug therapy in specified situations. For diseases such as Alzheimer's, cancer, AIDS, Amyotrophic lateralsclerosis [ALS], multiple sclerosis, cystic fibrosis, pneumonia, asthma, bronchial hyperresponsiveness and atopy, chronic obstructive pulmonary disease (“COPD”) and many bacterial infections, they are either caused by, or reflect an imbalance of redox and consequent glutathione depletion. That glutathione depletion is more indicative of a particular problem pointing to a particular therapy as described in this invention than diminished anti-oxidant capacity of the body as a result of disease. Similarly, the invention is useful in connection with uncontrolled macrophages usually manifested by exaggerated inflammatory response, such as septicemia and in mutomuscular diseases. Meningococcal infections are enhanced by the therapy of propensity and treatment under this invention. Even if not curative, restoration or amelioration of glutathione depletion is therapeutic. In the event of trauma, often the stress of trauma complicates other bodily functions. Restoration of the functionality of the glutathione cycle improves the efficacy of other therapy. Therapy to increase anti-oxidant capacity is also ameliorative.

A different and useful of this invention is determination by evaluation of DNA sequences of the therapeutic value of vaccination. Those with “weaknesses, ” for instance, in the GSM (glutathione-S-transferase mu, also called “GST-mu” or “GSTM”) genotypic expression, suggesting a propensity to glutathione depletion and redox imbalance will be more susceptible to aggravated symptoms and deleterious effects of diseases. If a vaccine exists for such a disease, a vaccine would be more therapeutic for a person with such a propensity to glutathione depletion and redox imbalance. An example is Group B meningococcus (Neisseria meningitis) where a patient with propensities reflected in GSM genotypic expression to glutathione depletion and would find a vaccine more therapeutic than a person without such a propensity. If there was a vaccine risk, those who had a GSM defect would be more likely to take the vaccine risk, rather than face the increased risk from less effective immune response to Group B meningococcus that results from a GSM defect.

For military personnel faced with close living, or with vaccination for anthrax or other biological warfare agents, DNA testing for GSM or CYP450 defects would be important to pre-select personnel more prone to complications, who could be pre-administered cystine or other glutathione pathway enhancing and detoxifying compound as later discussed.

Having rendered the sequence(s) of DNA alleles from a client or patient in electronically readable form, such as by a Perkin-Elmer/PE Biosystems model ABI PRISM 377 DNA Sequencer, U.S. Pat. No. 5,543,026, Hoff et al, Aug. 6, 1996, and associated software sold with that instrument in electronically readable form, the inventors could be electronically examining the sequences stored for a biochemical defect suggestive of redox imbalance. That examination would be done by comparing the sequences of a particular client with a data base of known sequences of alleles reflecting potential risks or actual alleles found with respect to specific diseases or afflictions.

Previously, the direct attack of gene by gene, allele by allele comparison, a massive investigation of DNA sequences has been impractical because of the limited speed of general purpose computing machines, limited storage capacity, and insufficient knowledge of the complete human DNA chain. The optimal means of investigation is to examine DNA sequences on chromosomes known to evidence genotypic expression of concern, as opposed to examining the entire DNA sequence in every instance. See, Fryer A A, Bianco A, et al, Am J. Respirator and Critical Care Medicine May 2000, 161(5): 1437-42. Recent enhancements in the speed of generally available computing machines, such as 1300 Mhz, and terabyte storage render massive analysis practical. The compilation of the human genome sequence enables practical comparison and knowledge of DNA sequences suggesting propensity to various afflictions, examples of which are cited in this invention, though the invention and method is not limited to the specific examples cited.

As previously stated, the invention is a system of proposed treatment based on knowledge of the DNA sequence, comparison with databases of genotypic expression associated with various diseases and afflictions, and from there, when propensities are shown, investigation by examination of the glutathione pathway functionality and redox imbalance to determine further treatment, particularly treatment by cystine, to restore proper glutathione levels. For other genotypic expressions, other treatments to restore proper function of the glutathione pathway and redox potential are indicated, but in any event, knowledge of the DNA sequences and the determination of glutathione pathway functionality and/or redox imbalance would be made according to the methods in this invention. Nedecheva K, Andersen, T Erikstein B, Nesland et al, Institute for Cancer Research, The Norwegian Radium Hospital, Dept. of Genetics, Home Staff Scientific Interest Publications Links available on Internet

The addition of yet another step, testing and measuring the anti-oxidant capacity of the body is a further refinement of the invention as a diagnostic tool. Again, the entire population could be tested for anti-oxidant capacity of the body, but such testing is not cost-effective. The DNA sequence analysis, comparison with known sequences to determine genotypic expressions and disease propensity, and testing for glutathione deficiency as indicated and for anti-oxidant capacity, as indicated, enable sophisticated and earlier evaluation of patent and latent disease conditions.

The examination of the presence of certain genes as indicating a propensity to disease such as breast cancer is stated in the art. Based on the ability to determine DNA sequences, and the compilation of human genome sequences, the invention contemplates the novel step of identifying gene sequences to an individual, and then comparing the gene sequences with a database of known DNA sequences, and thereupon determining a propensity to a certain disease or disease state by storage and testing of DNA. The entire DNA need not necessarily be examined, for instance of an ancestor, until a necessary testing state is indicated, and the invention contemplates storage pending such need. Selection of key chromosomes and genetic sequences for deductive analysis of a patient's propensity based on known gene sequences indicating propensity is contemplated. The invention discusses later the development of such information and exemplary gene sequences to analyze.

Moreover, the invention recognizes that GST and GSM-μ1 defects are not the only significant genotypic expressions. Other genotypic expressions may lead to other defects in biochemistry and in particular biochemical cycles. This invention includes examination of other gene sequences to determine genetic propensity to disease and the examination of redox potential in applicable cycles, which will often and usually include the glutathione cycle. Upon such examination of redox potential, administration of therapy should be given to not only balance glutathione cycle misfunction, but also to ameliorate malfunction and redox imbalance in other cycles.

The method includes the additional possibility of drawing and analyzing samples drawn from grandparents, parents, siblings or other relatives to further clarify propensity. Such samples could be taken immediately after death in conjunction with funeral arrangements for the purposes of determining propensity and risk factors based on detected sequences synonymous or similar to known sequences or statistically apparent based on manifested symptoms in such relatives. Such determination of propensity in relatives of a particular client could be used to optimize selection of DNA sequences and alleles to study in a particular client.

Examination of critical DNA sequences of a patient's ancestors' prior to or contemporaneous to death while DNA is intact offers an opportunity to detect if genotypic DNA is inherited or is a result of an environmental influence or other mutation. Examination would first be made of corresponding genes which should be or could be present by inheritance, and then comparing gene sequences of a parent with a child to see if they properly coincide.

One of the most important databases of sequences to examine is a database of DNA sequences associated with glutathione S-transferase (“GST”). For example, by accessing a database having the sequences of glutathione S-transferase mu 1 (“GSTμ1”) defects, and electronically scanning the client's electronically stored DNA sequence, we can determine if GSTμ1 defects exist. Those defects, if present, indicate a genotypic predilection to many disease states. Other examples include examination for GSTP1, GST1 referenced in Curran, Weinstein, Griffiths, Cancer Letter May 29 2000, 153(1-2): 113-20. Other literature references five related gene classes to GST referenced as classes alpha, mu, pi, sigma, and theta. Hayes, Pulford, Critical Review Biochemistry Molecular Biology 30(6): 445-600 (1995). The level of expression of GST is asserted to be a crucial factor in determining the sensitivity of cells to a broad spectrum of xenobiotic (potentially toxic) chemicals. Overexpression in certain cases can be as deleterious as underexpression in others. Another example of the importance is cytochrome P450 2E1 (“CYP2E1”) a defect which is suggested to be associated to susceptibility to development of esophageal cancer. See, Tan W, Song N, Wang G Q, Lu A, Tang H J et al, Cancer Epidemiology Biomarkers Prev 2000 June, 9(6): 551-6. The cytochrome P450 DNA gene sequence will be generally referred to as CYP450.

The manifestation of those disease states is reflected in imbalance in redox potential. The method proposed in this invention enables potentially earlier detection of susceptibility to a particular disease state, earlier detection of latent onset of a disease, and as will be described, more accurate clinical therapy for a client manifesting symptoms of a disease.

In the situation of a GSTμ1 defect, and in actual observation of redox imbalance, a client would be consulting a health care provider for a protocol to restore redox potential and enable optimal administration of suitable supplements, ameliorative substances or drug therapy to optimize the glutathione pathway. For instance in situations where treatment would normally involve surgery of a manifestation of a disease, such as a tumor, or “watchful waiting” for manifestation and then treatment, the administration of cystine to increase the competency of the immune system prior to such manifestation and delay such manifestation results in reduced medical costs and increase in quality of life as well as reduction of the stress of “watchful waiting.” The invention contemplates that the determination of the propensity to glutathione deficiency would suggest more frequent testing and therapy to forestall glutathione therapy.

Several specific applications are as follows. For patients on a ventilator, the patient's DNA sequences could be compared to databases of defects indicating a propensity to redox imbalances in the glutathione pathway. In any event, for patients on a ventilator, determination would be made if there are redox imbalances which are associated with the glutathione pathway. A redox imbalance implies that peroxidative injury is occurring and the glutathione cycle is malfunctioning. Measurement of antioxidant capacity or for purposes of a patient with a problem, incapacity, showing a redox imbalance will be discussed later.

The importance is that glutathione can protect hemoglobin and other critical blood cell proteins from peroxidative injury. Glutathione reductase links the pathway to the hexose monophosphate pathway through the reversible oxidation and reduction of NADP. Glutathione peroxidase effects the conversion OH to water, thus reducing the likelihood of peroxidative denaturation of hemoglobin and other proteins.

### Glutathione Level Test

Determination of glutathione levels for plasma and/or red blood cells is the preferred test. Red blood cell glutathione measures oxidized and reduced glutathione in the red blood cells. The predominant form is the reduced form of glutathione. Plasma glutathione measures total glutathione plus all other thiols and thiol containing proteins present in plasma. If a separately performed assay of plasma glutathione is done by high performance liquid chromatography (HPLC), the amount determined by that test can be subtracted from plasma glutathione to yield total thiol. The test of glutathione is performed according to F. Tietze, 1968 Enzymic Method for the Quantitative Determination of Nanogram Amounts of Total and Oxidized Glutathione Analytical Biochemistry with an additional reference of F. Tietze, 2^{nd} ed., Chemical Chemistry 1994, pp. 1779-1780. This Tietze method has been modified as follows:

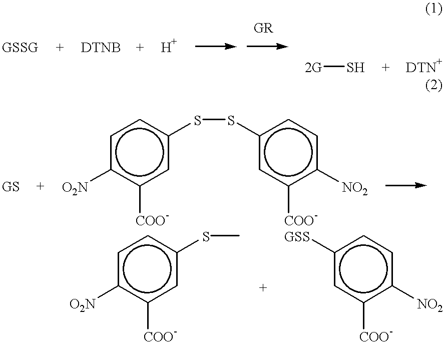

where GSSG is gluthathione, oxidized

GR is glutathione reductase

DTNB is a sulfhydryl reagent 5, 5¹-dithiobis-(2-nitrobenzoic acid)

G-SH is glutathione, reduced

DTN⁺ is dithiobisnitrobenzoic acid

GS is a transition state between glutathione reduced and oxidized

The method of glutathione assay provides a sensitive method for total and oxidized glutathione. The modification increases sensitivity for spectrophotometric analysis. The reagents in use throughout this invention, including for this test, are either generally available for a chemical supply house or available from Sigma Chemical Co., Inc. or a company associated with it, Aldrich Chemical Company, of St. Louis, Mo., incorporating DTNB, a sulfhydryl reagent 5, 5¹-dithiobis-(2-nitrobenzoic acid) in the first reaction which possesses a molar absorption at 412 mμ then forms two moles of GSH per mole of reduced nucleotide utilized in the GSSG reduction in reaction (2). The rate of chromophore development depends on the concentration of glutathione in the reaction mixture detectable to 10 nanograms ml⁻¹. This provides a highly sensitive and specific procedure for measuring glutathione. The normal level should be approximately 200-400micromoles/liter for plasma and red blood cells. The test may be performed on an automated clinical chemistry analyser (also called a random access analyzer) such as Roche Cobas Fara. Samples are collected carefully to prevent contamination. Frozen plasma collected from ACD, EDTA, and heparin may be used. The invention could test reduced glutathione but there is not any efficacy over testing total glutathione. Upon determining a redox imbalance or suboptimal glutathione cycle function, the next step in the preferred mode of treatment protocol would be administering cystine, normally in the amount of be 140 mg/70 Kg man twice per day. The cystine feeds into the glutathione pathway and substantially reduces a stay on a ventilator with substantial cost savings and therapeutic effect. A discussion of the therapeutic value of appropriate levels in the glutathione pathway is discussed in Rahman I, MacNee W, Free Radical Biological Medicine May 1, 2000, 28(9): 1405-1420, and in particular as associated with inflammatory lung diseases.

Cystine will be used as a generic reference for a glutathione pathway enhancing and detoxifying compound. Cystine is the preferred compound to be used as a glutathione pathway enhancing and detoxifying compound. Such glutathione pathway enhancing and detoxifying compounds include the following:

Cystine is (3,3′-dithiobis [2-aminopropanoic acid]). Cystine is readily reduced to cysteine. Cystine is present in most mammalian hair and keratin.

Cysteine is 2-amino-3-mercapto propanoic acid. It is readily converted by oxioreduction to cystine. It is a constituent of glutathione and abundantly present in the metallothioneines.

Cystine in the body-useful form as L-cystine is available from Spectrum Chemical Mfg. Corp. 14422 S. San Pedro St., Gardena, Calif. 90248.

Cystine, cysteine, and N-Acetyl cysteine and pharmaceutically acceptable salts, including the pharmaceutically active forms described in Kozhemyakin et al, published by WIPO as WO 00/031120, PCT/RU99/00453, filed internationally on Nov. 19, 1999, “Hexapeptide with the Stabilized Disulfide Bond and Derivatives Thereof Regulating Metabolism, Proliferation, Differentiation and Apoptosis,” will all collectively be referred to as cystine in this invention. Included in the term cystine is also any therapeutically beneficial sulfur donating compound, including ebselen, which interacts with the glutathione pathway. The invention contemplates in the term cystine undenatured whey protein products designed to have enhanced cystine concentration as well as protein products which contain cysteine and cystine. They can be in the form of food products. Immunocal® whey protein diet supplement by Immunotek Research Ltd. of Montreal Quebec is a useful product with cystine.

The addition of cystine, cysteine, N-acetyl cysteine, or the pharmaceutically acceptable salt of those substances yields another effect in this invention not facially evident from the independent properties of the basic components of the invention. Administration of a glutathione pathway enhancing and detoxifying compound, preferably cystine, which has the best and most rapid upload into the glutathione pathway and better storage capability by the body, or N-acetyl cysteine, enhances the immune system competency of the patient.

All of these cystine and cystine-like compounds function as a glutathione pathway enhancing and detoxifying compound. They have the additional benefit of ameliorating the negative renal, hepatic and gastric effects of COX-2 inhibitors and HMG-CoA inhibitors, both as a combination and individually. The enhancement of the glutathione level and pathway has a second important and unexpected effect. The avoidance of a glutathione deficiency steers the patient to have a higher Th-1 response to Th-2 response ratio than the patient would have with any glutathione deficiency.

Anti-oxidant capacity is best measured by measuring the capacity of human plasma. The recommended procedure is to measure the antioxidant capacity of plasma saliva and bronchoalveolar lavage fluid based on the absorbance of the ABTS⁺-. On exposure to hydrogen peroxide, metmyoglobin and methemoglobin are activated to ferryl states in which the iron is one oxidizing equivalent above the original level and one oxidizing equivalent is on the surface of the protein. With reducing agents, these species are reduced back to metmyoglobin or methemoglobin.

### Anti-Oxidant Capacity Test

The procedure has been applied to physiological antioxidant compounds and radical scavenging drugs. The basic principles of the procedure are as follows. An antioxidant ranking has been established based on their reactivity to a 1.0 mM/L TROLOX standard. The peroxidase activity of metmyoglobin combined with its interaction forms a radical cation intermediate with a phenothrozine compound. The method derives from the observation that when 2,2-azinobis-3-ethylbenzo thiazoline-6-sulphonic acid (ABTS) is incubated with peroxidase and hydrogen peroxide, the relatively long-lived radical cation, ABTS⁺- is formed. A large number of free radicals such as hydroxyl, peroxyl, alkoxyl and inorganic radicals also react rapidly with ABTS to form this species. When the peroxidase is metmyoglobin, the formation of the ABTS⁺- radical cation in interaction with ferrylmyoglobin has spectral absorption maxima at 650 nm, 734 nm and 820 nm, beyond the region of the heme proteins. In the presence of antioxidant reductants and hydrogen donors in plasma, the absorbance of this radical cation is quenched to an extent related to the antioxidant capacity.

The major antioxidant defenses in plasma include ascorbate, protein thiols, bilirubin, urate and α-tocopherol. The “chain-breaking” or “radical scavenging” agents against oxidative stress act in the above sequence of increasing or decreasing effectiveness against free radicals generated in the plasma aqueous phase. Plasma also contains the “preventive” antioxidants, ceruloplasmin and transferrin, the iron-scavenging proteins whose contribution to the overall antioxidant capacity is to prevent iron availability. Applying this method, the total plasma antioxidant status of an individual can be determined supporting the need for antioxidant supplementation as well as the monitoring of blood levels ranging from pregnancy (preterm infants), adults with COPD and asthma.

### Speciman Collection and Handling

Universal Precautions Apply.

Fresh plasma samples (EDTA or Citrated) should be used.)

See skin puncture collection of plasma, in Standard Laboratory Procedure Manual

See venipuncture collection of plasma in Standard Laboratory Procedure Manual

Blood should be collected asceptically and separated by standard laboratory techniques.

Contamination or introduced particulate matter can interfere with absorbance leading to erroneous results. Critically, it is the plasma anti-oxidant capacity being measured, not that of the white or red blood cells. That too can be measured, but is less reliable in certain circumstances.

### Storage of Sample

Samples can be stored at −20 degrees C. for up to three (3) months. Avoid repeated freeze-thaw cycles. Do not store samples in a self-defrosting freezer. If samples are not assayed within twenty four (24) hours of collection, the samples must be stored at −20 degrees.

### Materials and Equipment

### Test Instruments or Equivalent to be Utilized

Manual: Variable wavelength Spectrophotometer

Automated: Roche Cobas Fara

### Materials

1. Phosphate buffered saline (“PBS”), pH 7.4

2. Myoglobin (Molecular Weight [“MW”]18,800 or 17,600), 400 μM in PBS

3. Potassium Ferricyanide K₃Fe(CN)₆, MW 329.2, 740 μM in PBS

a. Prepare the double strength of the solution 2 and 3 Mix well and maintain at ambient temperature for 15 minutes.

b. Dialyse against 400 mL PBS at +4° C.; then re-dialyse with fresh buffer; change of buffer after 2 hour interval and last dialysis for 20-30 minutes.

c. Read the absorbance at 490, 560, 580 and 734 nm and calculate the metmyoglobin (MetMb) concentration using Whitburn's equation:

d. This solution is the stock solution and can be stored at −20degrees C. for up to 4 weeks.

e. Working solution: Dilute with appropriate volume of PBS to give a final MetMb concentration of 70 μmole/L-stable at +4 degrees C. for 1 week.

6. TROLOX® MW 250.29 [registered trademark of Hoffman LaRochel]: 97% fine white powder manufactured by Aldrich Chemical associated with Sigma Chemical Co. of St. Louis, Mo.

a. Do not use yellow lumps, use white fine powder.

b. Stock solution: 2.5 mM in PBS, solution to be sonicated at high speed, store at −20 degrees C. for up to 4 weeks

c. Working solution: 1:10 v/v with PBS, 0.25 mMole/L, stable 1 week at +4 degrees C.

d. Standard curve: 2.5 nanomole to 12.5 nanomole

5. ABTS-2,2¹-AZINOBIS (3-ETHYLBENZOTHRAZOLINE-6-SULPHONIC ACID) MW 548.7

a. Prepare FRESH—stable at ambient temperature for 24 hours, protect from light

b. Stock Solution: Prepare 5 MM (5 m Mole/L) solution in PBS

c. Working Solution: 1:10 dilution with PBS. (0.5 m Mole /L, stable at ambient temperature for 24 hours, protect from light).

4. H₂O₂ (30% w/w), MW −34.01, 9.8M

a. Prepare fresh.

b. Calculate the concentration of 30% solution by taking absorbance at 240 nm

c. Stock Solution 0.098M solution in PBS

d. Working Solution: 1:11 dilution with PBS (450 μmole /L)

### Procedure

1. To 1 mL polystyrene square curvette-1 cm path length-597 μL of PBS, pH 7.4

300 μL of ABTS solution

Desired volume of standards and unknown, and adjust the final volume with PBS:

36 μL of MetMb solution (70 μmole/L)

167 μL of H₂O₂ working solution.

1000 μL final volume

2. Use 10 μL to 50 μL: 2.5 n moles to 12.5 n moles to preapre a TROLOX standard curve.

3. 5-10 μL of plasma (on ice) (Heparinized). Mix well and invert by holding with parafilm and maintain for 12 minutes at ambient temperature and read immediately at 734 nm.

### Results

1. Plot a standard curve n moles/L TROLOX on x-axis; Absorbance reading at 734 nm on Y axis

2. Determine the TROLOX equivalent antioxidant capacity (“TEAC”) in Mm) by using linear regression analysis compared to known absorbance of TROLOX.

The normal range will be 1.2-1.6 milliMoles/liter. Patients exhibiting anti-oxidant capacities below 1.2 milliMoles/L demonstrate oxidative stress reflecting an imbalance in the redox system with a concomitant depletion of plasma glutathione. However, red blood cell and white blood cell glutathione may exceed the normal range as a result of the body's protective mechanisms. Administration of cystine to restore plasma glutathione levels is the efficacious and expeditious treatment.

Thus, propensity of glutathione imbalance by examination of a patient's DNA sequences, and a result indicating such propensity would suggest early intervention and aggressive investigation into redox imbalance and the state of the glutathione pathway.

For pregnant women, DNA sequencing would be performed and DNA sequences compared to a data base of sequences indicating a predisposition to preeclampsia. After comparing the DNA sequences to known defect sequences per the steps described, testing would be undertaken to determine if there are redox imbalances, including those associated with the glutathione pathway.

After such testing for propensity to preeclampsia, if the glutathione level was sub-optimal, the next step in a protocol of treatment would be administering cystine or other appropriate drug therapy. The cystine feeds into the glutathione pathway. Such treatment restores the health of the mother more rapidly, and such treatment, after determining redox imbalances in the baby, substantially reduces the time a baby delivered by a mother exhibiting symptoms or preeclampsia would remain in a neonatal intensive care unit.

Recently a patent issued for “Intravenous magnesium gluconate for treatment of conditions caused by excessive oxidative stress due to free radical distribution”, William B. Weglicki, U.S. Pat. No. 6,100,297, Aug. 8, 2000. The abstract refers to the intravenous use of magnesium gluconate to substantially block fee radical surge in the treatment of ischemia/reperfusion (I/R) injury due to oxidative stress.

The difference in this invention is that this invention proposes to first determine the genetic propensity to glutathione imbalance, which is associated with free radical surge in the treatment of ischemia/reperfusion injury due to oxidative stress. See, Mak et al, Circulation Research 70(6), June 1991, pp. 1099-1103. By testing for glutathione level, and by determining the anti-oxidant capacity of the patient, the necessity and acuity of condition for determination of use of the Weglicki invention can be made.

The inventors also propose combination in sequence of devices associated with one or more general purpose computers that have means for performing the above steps. The invention contemplates a combination of a DNA sequence analyzer, and automated clinical chemistry analyzer or random access biochemical analyzer such as the Roche Cobas Fara and a general purpose computing machine having either a database of known genetic sequences and associated propensities, and/or an additional data base of redox potentials and glutathione levels associated with one or more particular diseases.

Potentially continuous sampling to automate and provide feedback to optimize therapy with respect to detected redox imbalances and/or suboptimal glutathione levels is also contemplated using the combination (without the DNA sequencer, which may or may not have been previously done once a disease is manifested) in the prior paragraph. Upon restoration of proper glutathione level, the therapy for biochemical balance could be automatically discontinued.

The foregoing contemplates a deductive method of treatment based on DNA sequences, genetic propensities and determination of redox imbalance and glutathione level.

The invention also claims an inductive use of the invention. By analyzing redox imbalance, and glutathione level in combination with actual disease manifestations, and then obtaining DNA sequences of patients with such characteristics, existing databases can be enhanced, or new databases developed. With the ability to actually review the entire human genome sequence, statistically significant correlations by standard statistical methods, especially using statistical software packages such as those available from SASS (Reg. Mark) can be used to associate DNA sequences with redox imbalance and glutathione level as to certain disease manifestations. Such database can then be utilized as the database of genotypic expression to compare with an unknown patient to determine that patient's propensity to particular disease. A corollary of such a database is the ability to associate redox imbalance and glutathione level in certain disease manifestations with severity or advancement of disease.

Returning to the ventilator example, such a database would enable a patient on a ventilator who had certain genotypic propensities to be evaluated, based on redox potential and glutathione level for acuity of the situation. As a further part of that combination, an instrument for determining redox imbalances in a patient could be integrated with the combination in the prior paragraph to enhance and statistically adjust or build a data base to determine propensity based on actual redox imbalances, and die reappearance of glulathione deficiency associated with gene sequences, and the database could be examined for other statistically significant sequence coincidences.

For the preeclampsia example given, development of a database of known DNA sequences so determination can be made of propensity to preeclampsia enables a health care provider to test for redox imbalance and glutathione deficiency and make early determination of adverse bodily state and acuity of any adverse condition.

Enhancement of a database would be done by having an instrument analyze DNA sequences, and software associated with such instrumentation that then complies the information into electronically readable form. The novel combination would be to combine the electronically readable information with software installed on the same or a linked general purpose computer which software would perform an automated comparison with a data base of known sequences indicating propensity to a particular redox imbalance and substitute in further information to alter the database, which as the data base grows, artificially enhances the intelligence of the database for diagnostic purposes.

In a sense, two related databases are developed. The first is a database for gene sequences on certain chromosomes for indicating propensity, and enabling selection of critical DNA sequences to examine, and the second database is, given a propensity and a certain redox potential, glutathione level, and alternatively or additionally, determination of the anti-oxidant capacity, what is the state of the manifestation, if any, of the indicated propensity. The anti-oxidant capacity can be a predictive tool for likelihood of manifestation or stage of disease illustrating the presence of disease symptoms, but glutathione is equally and more precisely determinative.

Better yet is to have additional fields in the second database with objective patient data such as blood pressure, temperature, blood analysis data, cholesterol, blood cell counts, cytokine levels, and additional fields with less objective/more subjective data that can be electronically manipulated such as Karnofsky performance criterion, or WHO recommendations for grading of acute and subacute toxicity. Outcome data can be based on WHO recommendations for grading of acute and subacute toxicity, including days/time to change as a result of therapy in a patient. Many of these objective criteria can be seen in Provisional application 60/297,816 filed Jun. 13, 2001, and Provisional Application Application 60/263,486 filed Jan. 23, 2001 both entitled “Adjuvant Immune Therapy in the Treatment of Solid Tumors through Modulation of Signaling Pathways following Engagement of Humoral and Cell Mediate Responses.” The data base can be used to evaluate treatment methodologies given objective criterion, and as more patients are entered in the data base, even if not accompanied by al the same fields of data, the data base can be evaluated correlation between selected data and certain treatment methodologies if patient data is not provided. As the database develops, missing data which is statistically determined to be irrelevant to the selection of treatment would be output and so indicate irrelevancy. For instance, body temperature may have nothing to do with propensity to cardiac event as shown by lack of correlation between body temperature and historical occurrence of cardiac event. Further body temperature may not statistically correlate with treatment methodology in the historical patient data base. Thus, failure to have such data when presented with a cardiac patient with a particular genetic sequence would not affect a proposed patient's treatment and the output would indicate the missing data is not critical.

The feature of a directed course of treatment methodology for a certain patient profile of fields of data can also be added, particularly at the early stages of the development of the database.

The invention contemplates that a genotypic propensity may be seen in DNA sequences of an ancestor and a patient-child of such ancestors. However, such propensity may be unlikely to manifest if antioxidant capacity of the body is high and glutathione level is normal. At the same time, a given environmental factor may aggravate propensity. By using this invention, a health care provider can provide appropriate medical advice, and preventative therapy to maintain antioxidant capacity and properly glutathione cycle function.

Even for incurable diseases, monitoring of redox imbalance and glutathione function can ameliorate or arrest deleterious effects. The invention proposes to evaluate the propensity of a patient with an apparently incurable disease to glutathione cycle malfunction, and evaluate antioxidant capacity, and then administer therapy to restore normal glutathione levels and redox potential, and enhance antioxidant capacity.

An example of the reference in the claims to “database capable of generating output from the electronic results of a clinical chemistry analyzer and DNA sequencing machine is the database described in the prior paragraphs. A combination of a DNA sequencing machine, a clinical chemical analyzer such as one made by Roche Cobas Faras, both generating electronic output, and a general purpose computer and the database discussed could generate preferred treatment methods and expedite a health care providers' selection, and enable delegation of proposed treatment to less skilled personnel.

The steps in the claims as a description of the practice of the invention and are incorporated by reference. The claims set out the programming steps and modules needed. A variety of database management languages can be used as well as any number of database management packages known to those skilled in database management arts and analysis.

Electronic security measures (normally a password) are contemplated as accompanying data sets or data bases referenced in this invention.

The invention is not meant to be limited to the disclosures, including best mode of invention herein, and contemplates all equivalents to the invention and similar embodiments to the invention for humans and animals and veterinary science. Other tests can determine glutathione level, redox imbalance, and anti-oxidant capacity, and are contemplated in the invention and claims.

1. A method of treatment of a patient having certain genetic defects, the method comprising the following steps:
making a DNA sequence determination if a patient has at least one genetic defect selected from the class of GST defects or CYP450 defects indicating a propensity to redox imbalance; and
upon said determination, administering a glutathione pathway enhancing and detoxifying compound to said patient.

2. The method according to claim 2, further comprising the following step:
recording said DNA sequence determination onto electronic media for storage.

3. The method according to claim 3, further comprising the following step:
Connecting translation software to said electronic media having said DNA sequence determination and in said translation software inserting hyperlinks to a data base for information concerning any abnormal DNA sequence to readily access further information concerning any said abnormal DNA sequence.

4. The method according to claim 4, further comprising the following step:
incorporating an electronic security system into said electronic media.

5. The method according to claim 1, further comprising the following step:
Connecting translation software to said electronic media having said DNA sequence determination and further connecting output from said translation software to software containing hyperlinks with said DNA sequence determination to a data base for information concerning any abnormal DNA sequence to readily access further information concerning any said abnormal DNA sequence.

6. The method according to claim 5, further comprising the following step:
incorporating an electronic security system into said electronic media.

7. The method according to claim 1, further comprising:
subsequent to making a DNA sequence determination if a patient has said at least one genetic defect, testing said patient's anti-oxidant capacity.

8. The method according to claim 1, further comprising:
subsequent to making a DNA sequence determination if a patient has said at least one genetic defect, testing said patient's total thiol.

9. The method according to claim 1, further comprising:
subsequent to making a DNA sequence determination if a patient has said at least one genetic defect, testing said patient's glutathione level.

10. A method of treatment of a patient having certain genetic defects, the method comprising the following steps:
determining if a patient has at least one genetic defect selected from the class of GST defects or CYP450 defects; and
testing said patient's anti-oxidant capacity;
upon testing said patient's anti-oxidant capacity, if said anti-oxidant capacity is inadequate, administering a glutathione pathway enhancing and detoxifying compound to said patient.

11. A method of treatment of a patient having certain genetic defects, the method comprising the following steps:
determining if a patient has at least one genetic defect selected from the class of GST defects or CYP450 defects;
testing said patient's total thiol;
upon testing said patient's total thiol, if said total thiol shows glutathione pathway impairment, administering a glutathione pathway enhancing and detoxifying compound to said patient.

12. A method of treatment of a patient having certain genetic defects, the method comprising the following steps:
determining if a patient has at least one genetic defect selected from the class of GST defects or CYP450 defects;
testing said patient's glutathione level;
upon testing said patient's glutathione level, if said glutathione level is inadequate, administering a glutathione pathway enhancing and detoxifying compound to said patient.

13. A method of enabling a person to determine propensity to disease caused from xenobiotic influences, the method comprising the following steps:
making a present at least one DNA sequence determination from said person;
recording said present at least one DNA sequence determination onto electronic media for storage;
comparing said present at least one DNA sequence determination electronically with a known data base to make a determination if a defective DNA sequence is present;
making a former DNA sequence determination of said at least one DNA sequence from said person from an earlier point in life;
recording said former DNA sequence determination onto electronic media for storage;
comparing said former DNA sequence determination with said person's present DNA sequence to determine if xenobiotic alteration of at least one cell has occurred causing said present defective DNA sequence.

14. A method of enabling a person to determine propensity to disease caused from inherited influences for at least one DNA sequence, the method comprising the following steps:
making a present at least one DNA sequence determination from said person;
recording said present at least one DNA sequence determination onto electronic media for storage;
comparing said present at least one DNA sequence determination electronically with a known data base to make a determination if a defective DNA sequence is present;
making a former DNA sequence determination of said at least one DNA sequence from said person from an earlier point in life;
recording said former DNA sequence determination onto electronic media for storage;
comparing said former DNA sequence determination with said person's present DNA sequence to determine if xenobiotic alteration of at least one cell has occurred causing said present defective at least one DNA sequence;
making an ancestral DNA sequence determination of said at least one DNA sequence from at least one ancestor of said person;
recording said at least one DNA sequence determination onto electronic media for storage;
comparing said ancestral DNA sequence determination with said former DNA sequence determination and with said person's present DNA sequence determination to determine if xenobiotic alteration of at least one cell has occurred or if said present defective DNA sequence is inherited.

15. The method according to claim 14, further comprising the following step:
Connecting translation software to said electronic media having said DNA sequence determination and in said translation software inserting hyperlinks to a data base for information concerning any abnormal DNA sequence to readily access further information concerning any said abnormal DNA sequence.

16. The method according to claim 15, further comprising the following step:
incorporating an electronic security system into said electronic media.

17. The method according to claim 14, further comprising the following step:
Connecting translation software to said electronic media having said DNA sequence determination and further connecting output from said translation software to software containing hyperlinks with said DNA sequence determination to a data base for information concerning any abnormal DNA sequence to readily access further information concerning any said abnormal DNA sequence.

18. The method according to claim 17, further comprising the following step:
incorporating an electronic security system into said electronic media.

19. A method of evaluating patient condition, the method comprising the following steps:
determining if a patient has at least one genetic defect by electronically comparing said patient's DNA sequence with known non-defective DNA sequences and generating electronic output of said at least one genetic defect;
connecting translation software to said output of said at least one genetic defect;
compiling into electronic form said patient's symptom data and objective condition data;
electronically correlating said patient's symptom data and objective condition data with said output of said at least one genetic defect;
generating risk data output from electronically correlating said patient's symptom data and objective condition data with said output of said at least one genetic defect to calculate potential risk data for said patient from said patient's DNA sequence and said patient's symptom data and objective condition data.

20. The method according to claim 19, further comprising:
based on said risk data output for said patient from said DNA sequence and said patient's symptom data and objective condition data, administering a prophylactic medication to said patient.

21. The method according to claim 20, further comprising:
further connecting said output of said at least one genetic defect from said translation software to electronic media containing hyperlinks with said DNA sequence determination to at least one data base for information concerning any abnormal DNA sequence to readily access further information concerning any said abnormal DNA sequence.

22. The method according to claim 19, further comprising:
compiling output from electronically correlating said patient's symptom data and objective condition data with output from said translation software of said DNA sequence determination;
based on output from compiling output from electronically correlating said patient's symptom data and objective condition data with output from said translation software of said DNA sequence determination, generating hyperlinks to a data base for information concerning interplay of any abnormal DNA sequence and said patient's symptom data and objective condition data.

23. The method according to claim 22, further comprising the following step:
incorporating an electronic security system into said electronic media.

24. A method of determination of propensity to complications from pregnancy based on certain genetic defects, and prophylactic treatment of a pregnant patient having certain genetic defects, the method comprising the following steps:
making a determination if a pregnant patient has at least one genetic defect selected from the class of GST defects or CYP450 defects; and
upon making said determination, administering as a prophylactic a glutathione pathway enhancing and detoxifying compound to said patient.

25. A method of determination of propensity to complications from a particular vaccine based on certain genetic defects, and prophylactic treatment of a patient proposed for a vaccine having certain genetic defects, the method comprising the following steps:
making a determination if a patient proposed to be administered a vaccine has at least one genetic defect selected from the class of GST defects or CYP450 defects; and
upon making said determination, administering as a prophylactic a glutathione pathway enhancing and detoxifying compound to said patient.

26. A method of determination of propensity to complications from COPD based on certain genetic defects, and prophylactic treatment of a COPD patient having certain genetic defects, the method comprising the following steps:
making a determination if a COPD patient has at least one genetic defect selected from the class of GST defects or CYP450 defects; and
upon making said determination, administering as a prophylactic a glutathione pathway enhancing and detoxifying compound to said patient.

27. A method of extending the life of a terminally ill patient having certain genetic defects, and prophylactic treatment of said patient while maintaining relatively better quality of life for said patient, the method comprising the following steps:
making a determination if a terminally ill patient has at least one genetic defect selected from the class of GST defects or CYP450 defects; and
upon making said determination, administering as a prophylactic a glutathione pathway enhancing and detoxifying compound to said patient.

28. The method according to claim 27, further comprising the following step:
monitoring the total thiol of said patient, and upon increase in sulfur excretion, augmenting the dose of said glutathione pathway enhancing and detoxifying compound to said patient.

29. A combination for evaluating a proposed treatment course for a patient comprising:
a DNA sequencing machine for determining at least if a patient tissue sample has a GST defect;
a clinical chemistry analyzer for determining chemical levels in said patient from a tissue sample;
a general purpose computer having a database capable of generating output from the electronic results of said clinical chemistry analyzer and said DNA sequencing machine to enable a health care professional to evaluate a patient's disease state and preferred treatment.

30. A method of creating probable outcome data for a particular patient using DNA sequence data and patient data based on prior patient experience, the method comprising:
obtaining in electronic form at least one DNA sequence data from a patient;
reducing to a standard coding system in electronic form a selected data set for each said patient of patient symptoms and collecting in electronic form objective patient data;
reducing to a standard coding system in electronic form treatment methodology for said patient;
reducing to a standard coding system in electronic form empirical outcomes for said patient;
inputting said at least one DNA sequence data, said selected data set, said treatment methodology, and said empirical outcome into storage media accessible from a general purpose computer;
arranging said at least one DNA sequence data, said selected data set, said treatment methodology, and said empirical outcome into a database so that upon electronic entry for a given proposed patient for whom said at least one DNA sequence data, patient symptoms and objective patient data are known, a table having one axis of empirical outcomes and a second axis of proposed treatment methodologies is generated with a frequency count for each empirical outcome given a proposed treatment methodology from which a most favorable proposed treatment methodology can be selected for a particular patient.

31. The method according to claim 30, enabling artificially intelligent determination of most favorable treatment methodology, further comprising the following steps:
weighting by an objective score said empirical outcomes in a desired order;
subsequent to inputting said at least one DNA sequence data, said selected data set, said treatment methodology, and said empirical outcome into storage media accessible from a general purpose computer, applying a statistical regression analysis algorithm to identify the most favorable outcome for each said treatment methodology for a given set of DNA sequence data, patient symptoms and objective patient data;
utilizing said most favorable outcome for each said treatment methodology for a given set of DNA sequence data, patient symptoms and objective patient data to select and generate a most favorable treatment methodology for a given set of DNA sequence data, patient symptoms and objective patient data;
so that upon entry for a particular patient of DNA sequence data, patient symptoms and objective patient data, output is generated of at least one recommended treatment methodology which is most favorable based on statistical analysis of all database patients' DNA sequence data, patient symptoms and objective patient data.

32. The method according to claim 31, further comprising:
inserting a pre-directed treatment methodology into said database for at least one set of DNA sequence data, patient symptoms and objective patient data.

33. The method according to claim 32, said method further comprising:
said DNA sequences being all GST DNA sequences and CYP450 sequences.

34. The method according to claim 31, further comprising:
for each treatment methodology that is not pre-directed, analyzing each said DNA sequence data, patient symptoms and objective patient data to examine if each said DNA sequence data, patient symptoms and objective patient data affected said empirical outcome for all patients in a statistically significant way by applying a statistical regression software package to analyze for statistically significant variation in said empirical outcome for each said treatment methodology for a change in at least any one of said DNA sequence data, patient symptoms and objective patient data;
upon output indicating a statistically significant variation in outcome for said at least any one of each said DNA sequence data, patient symptoms or objective patient data, and upon failure to provide for a particular patient at least some part of DNA sequence data, patient symptoms and objective patient data in said database, output is generated indicating recommended treatment methodology which is most favorable for said particular patient and also indicating that statistically significant data is missing in order of priority of most significant to least significant which may affect outcome for the recommended treatment methodology to enable a health care provider to obtain said missing data.

35. The method according to claim 34, further comprising:
inserting a pre-directed treatment methodology into said database for at least one given set of DNA sequence data, patient symptoms and objective patient data.

36. The according to claim 35, said method further comprising:
said DNA sequences being all GST DNA sequences and CYP450 sequences.

37. A method of enabling rapid consideration of the impact of genetic characteristics by a person, including a health care provider, the method comprising the following steps:
electronically accessing at least one selected DNA sequence for a person;
recording a data base of hyperlinks to information concerning said at least one DNA sequence;
enabling access to a data base of comparative DNA sequences so that any abnormalities in said at least one selected DNA sequence can be ascertained;
recording translation software for reading said at least one DNA sequence and for linking said at least one DNA sequence determination to said data base of hyperlinks so that said person, can utilize said hyperlinks for said person's at least one selected DNA sequence to access medical information concerning said at least one selected DNA sequence.

38. The method according to claim 34, said method further comprising:
said DNA sequences being at least one of the GST DNA sequences and CYP450 sequences.

39. A combination enabling rapid consideration of the impact of genetic characteristics by a person, including a health care provider, the combination comprising:
means for determining at least one selected DNA sequence for a person;
means for recording said at least one DNA sequence onto electronic media for permanent storage;
means for recording a data base of hyperlinks to information concerning said at least one DNA sequence;
means for enabling access to a data base of comparative DNA sequences so that any abnormalities in said at least one selected DNA sequence can be ascertained;
means for recording translation software for reading said at least one DNA sequence and for linking said at least one DNA sequence determination to said data base of hyperlinks so that said person can utilize said hyperlinks for said person's at least one selected DNA sequence to access medical information concerning said at least one selected DNA sequence.

40. The combination according to claim 36, said combination further comprising:
said DNA sequences being at least one of the GST DNA sequences and CYP450 sequences.
